(19) United States
(12) Patent Application Publication
(10) Pub. No.: US 2006/0246098 A1
(43) Pub. Date: Nov. 2, 2006

(54) Stable aqueous-based emulsion formulation comprising urea and salicylic acid and method of using same

(76) Inventors: Srinivasa Rao, Valley Stream, NY (US); Suresh Dixit, Fort Worth, TX (US); Folusho Obe, Middletown, NY (US); Avraham Yacobi, Englewood, NJ (US)

Correspondence Address: TARO PHARMACEUTICALS U.S.A., INC.;C/O VENABLE LLP, P.O. BOX 34385, WASHINGTON, DC, 20045-9998, US

(21) Appl. No.: 11/377,513

(22) Filed: Mar. 16, 2006

Related U.S. Application Data
(60) Provisional application No. 60/662,508, filed on Mar. 16, 2005.
(60) Provisional application No. 60/748,048, filed on Dec. 7, 2005.

(51) Int. Cl. A61K 31/7072; A61K 31/7048; A61K 31/655; A61K 31/573; A61K 31/496; A61K 31/4178

(52) U.S. Cl. 424/401; 514/171; 514/49; 514/254.07; 514/397; 514/150; 514/458; 514/474; 514/27; 514/536

(57) ABSTRACT

The present invention relates to a stable aqueous-based emulsion formulation comprising urea and salicylic acid and a method of preparing the same. In particular, the present invention relates to a method of treating a skin condition comprising the step of topical administering a stable form of a formulation comprising urea and salicylic acid.

### CROSS REFERENCE TO RELATED APPLICATIONS

This application claims the benefit under 35 U.S.C. §1.119(e) of Provisional Application Ser. Nos. 60/662,508 filed on Mar. 16, 2005, and 60/748,048 filed on Dec. 7, 2005, the disclosure of which is incorporated by reference in its entirety herein.

### FIELD OF THE INVENTION

The present invention relates to a stable aqueous-based emulsion formulation comprising urea and salicylic acid and a method for preparing the stable aqueous-based emulsion formulation. In addition, the present invention relates to a method of treating dry skin conditions using same.

### BACKGROUND OF THE INVENTION

Skin is composed of an epidermis and a dermis. The outermost layer of the epidermis is called stratum corneum. Stratum corneum consists of constantly renewing keratinized cells. A dynamic balance is maintained between desquamation and regeneration from the underlying epidermal cells. Normal skin has multiple functions such as serving as a diffusion and mechanical barrier. Alteration in the stratum corneum, such as hyperkeratosis and parakeratosis, may result in thickened and inflexible skin. The accumulation of retained keratinous material can result in skin inflexibility, scale formation, and a dry and rough texture. This altered skin results in dry skin conditions ranging from mild to severe, such as chapping, xerosis, hyperkeratosis, parakeratosis, psoriasis, ichthyosis, and atopic dermatitis. Effective treatment to relieve or prevent the symptoms of the dry skin conditions requires moisturizing and exfoliating of the altered keratinized cells.

Urea is a moisturizing agent that is used to hydrate and soften thickened corneous layers of the skin. U.S. Pat. No. 5,629,344 discloses using urea in an ophthalmic preparation to enhance hydration and soften keratin. Urea is used either in an aqueous-based solution or in the form of a cream (emulsion of the oil-in-water type), but such use has disadvantages due to the instability of urea that readily decomposes in aqueous solution to form carbon dioxide and ammonia. U.S. Pat. No. 3,666,863 discloses a formulation containing urea and lactic acid. Incorporation of lactic acid is shown to decrease the tendency of urea decomposition. While these formulations may relieve discomfort of associated scales and cracks, formulations containing urea alone appear not to be effective in correcting the underlying keratinization effects.

U.S. Pat. No. 3,879,537 discloses a formulation for controlling keratinization using alpha-hydroxy acids, keto acids and esters thereof. The composition contains at least one organic acid or ester thereof having two to six carbon atoms and having α-carbon functionality which is effective to promote healing of ichthyotic skin lesions. The composition is available in ointment base such as hydrophilic ointment or petrolatum. Dermal Therapy Urea Cream sold by Dermal Therapy Research Inc, (London, Ontario) contains urea and an alpha-hydroxy acid. While alpha-hydroxy acid penetrates deeper in skin layers to provide anti-aging and anti-wrinkle benefits due to its small molecular size, it does not stay long on a skin and therefore it is not an ideal exfoliant for use on the skin. Exfoliation involves removing the dead and damaged skin cells so that the outer layer of new skin becomes visible.

Salicylic acid (2-hydroxy benzoic acid or orthohydroxy benzoic acid) is a beta-hydroxy acid that acts as an ideal keratolytic agent (exfoliant). Keratolysis makes skin feel softer and smoother. A beta-hydroxy acid is a larger molecule than that of alpha-hydroxy acid (for example salicylic acid (which is a beta-hydroxy acid) has a molecular weight of 138.12; and glycolic acid (which is an alpha-hydroxy acid) has a molecular weight of 76.05. The larger molecular size enables the beta-hydroxy acid to remain on the skin surface relatively longer, and permit the keratolytic agent to more effectively penetrate and exfoliate within the skin pores. The action within the pores makes it an ideal exfoliant for use on the skin. In addition, beta-hydroxy acids often are lesser irritants as compared to alpha-hydroxy acids, making them welcome alternatives for sensitive skin subjects.

Several commercial products, such as Exfol Cream sold by Skin Biology (Bellevue, Wash.), Dermarest® Psoriasis Medicated Moisturizer sold by Del Pharmaceuticals, Inc., (Farmingdale, N.Y.), Betahydroxy Acid Cream sold by Reviva Labs (Hoddonfield, N.J.) and Salex™ Cream sold by Healthpoint, Ltd. (Forth Worth, Tex.) all contain salicylic acid as the sole keratolytic agent. While claim to be effective in keratolysis, these products suffer severe drawbacks as they do not contain urea sufficient to provide a moisturizing action. In addition, the amount of salicylic acid in these products is low (2%-6%), thus providing a sub-optimal exfoliation activity.

U.S. Pat. No. 5,776,920 discloses a formulation containing urea, lactic acid and salicylic acid in a moisturizing base for topical treatment of psoriasis. The composition is prepared in a neutral, hydrophilic cream containing, at least, water, sodium lauryl sulfate and petrolatum.

Ointment formulation comprising urea and salicylic acid is currently commercially available, and sold under the trade name Kerasal®. It uses a soft white petrolatum as base. Urea and salicylic acid have excellent stability in the petrolatum base. Notwithstanding its clinical advantages, the product is, however, greasy. The greasy nature of the ointment formulation restricts its usage often to night-time applications and compromises treatment compliance because of its readily adsorption on to the patients' apparel.

Aqueous-based emulsion formulation comprising urea and salicylic acid is available, such as Calicylic Crème sold by Gordon Laboratories (Upper Darby, Pa.). The formulation contains urea and salicylic acid in which water is used predominantly as a base. However, the cream begins to deteriorate (i.e., emulsion breaks down) within 1-5 minutes when subjected to storage temperature of 40° C. (i.e., accelerated storage condition), indicating unsuitability of the product.

There is a continuing need for an aqueous-based emulsion formulation containing urea and salicylic acid that is stable. Prior art endeavors in this field have fallen short because no stable aqueous-based emulsion formulation comprising urea and salicylic acid has been identified. The present invention meets this need by providing a stable, non-greasy aqueous-based emulsion formulation comprising urea and salicylic acid that is effective in treating dry skin conditions.

### SUMMARY OF THE INVENTION

It is an object of the present invention to provide a stable aqueous-based emulsion formulation.

In one aspect, the present invention provides a stable aqueous-based emulsion formulation, comprising:
(i) from about 8 to about 15 wt % moisturizing agent selected from the group consisting of urea, glycerin, sorbitol and propylene glycol;
(ii) from about 6 to about 15 wt % keratolytic agent selected from the group consisting of salicylic acid and benzoyl peroxide;
(iii) from about 15 to about 50 wt % water;
(iv) from about 2 to about 25 wt % emulsifying agent selected from the group consisting of a mixture of glyceryl stearate and PEG 100 stearate, a mixture of glyceryl stearate and PEG 75 stearate, a mixture of glyceryl stearate and PEG 30 stearate, and polawax; and
(v) from about 0.1 to about 10 wt % stiffening agent selected from the group consisting of cetyl alcohol, stearyl alcohol, cetostearyl alcohol and cetyl palmitate,
wherein said formulation is non-greasy when applied to skin and exhibits the following characteristics when subject to storage condition of at least about two weeks and at about 35° to about 40° C.:
   (1) physical appearance of emulsion does not change;
   (2) amount of the moisturizing agent does not change; and
   (3) amount of the keratolytic agent does not change.

In another aspect, the present invention provides a stable aqueous-based emulsion formulation, comprising:
(i) from about 8 to about 15 wt % moisturizing agent selected from the group consisting of urea, glycerin, sorbitol and propylene glycol;
(ii) from about 6 to about 15 wt % keratolytic agent selected from the group consisting of salicylic acid and benzoyl peroxide;
(iii) from about 15 to about 50 wt % water;
(iv) from about 2 to about 25 wt % emulsifying agent comprising a mixture of glyceryl stearate, PEG 100 stearate, tribehenin, cetearyl alcohol, PEG-20 cetyl phosphate, diacetyl phosphate, cetyl palmitate and polawax; and
(v) from about 10 to about 40 wt % solubilizer selected from the group consisting of hexylene glycol, Di-PPG2-myreth-10-adipate, and polysorbate 80, wherein said formulation is non-greasy when applied to skin and exhibits the following characteristics when subject to storage condition of at least about two weeks and at about 35° to about 40° C.:
   (1) physical appearance of emulsion does not change;
   (2) amount of the moisturizing agent does not change; and
   (3) amount of the keratolytic agent does not change.

It is another object of the present invention to provide a stable aqueous-based emulsion formulation further comprising an anti-fungal agent.

It is another object of the present invention to provide a stable aqueous-based emulsion formulation further comprising a corticosteroid.

It is another object of the present invention to provide a stable aqueous-based emulsion formulation further comprising a macrolide lactone.

It is another object of the present invention to provide a stable aqueous-based emulsion formulation further comprising an antioxidant.

It is another object of the present invention to provide a stable aqueous-based emulsion formulation further comprising an anesthetic agent.

It is another object of the present invention to provide a stable aqueous-based emulsion formulation further comprising a vitamin D₃ derivative.

It is another object of the present invention to provide a stable aqueous-based emulsion formulation further comprising an anti-viral agent.

It is another object of the present invention to provide a method of preparing the stable aqueous-based emulsion formulation.

In one aspect, the present invention provides a method for preparing a stable aqueous-based formulation, comprising the steps of:
a) combining an emulsifying agent with a stiffening agent to form a first solution;
b) dissolving a moisturizing agent in water to form a second solution;
c) adding the second solution to the first solution to form an emulsion; and
d) adding a keratolytic agent to the emulsion to form a stable aqueous-based formulation.

In another aspect, the present invention provides a method for preparing a stable aqueous-based formulation, comprising the steps of:
a) preparing a first solution of emulsifying agent;
b) dissolving a moisturizing agent in water to form a second solution;
c) adding the second solution to the first solution to form an emulsion;
d) adding a keratolytic agent to a solubilizer to form a third solution; and
e) adding the third solution to the emulsion to form a stable aqueous-based formulation.

It is another object of the present invention to provide a method for treating a skin condition comprising the step of administering a stable aqueous-based emulsion formulation comprising urea and salicylic acid.

### DETAILED DESCRIPTION OF THE INVENTION

Definitions

As used herein, the definition of terms are listed as follow:

“moisturizing” refers to a process of adding moisture to the skin (i.e., providing hydration to the skin);

“moisturizing agent” refers to an agent that performs the moisturizing process when applied onto the skin;

“keratolysis” refers to a process of removing the dead skin containing keratinous tissues from the skin. For the purposes of the present application, keratolysis (similar to exfoliation, see below) refers to a process that makes new skin feel softer and smoother;

“keratolytic agent” refers to an agent that performs the keratolysis when applied on the skin;

“exfoliation” refers to a process of detachment and shedding of superficial cells of an epithelium or from any tissue surface or scaling or desquamation of the horny layer of epidermis. For purposes of the present application, “exfoliation” refers to the same process as “keratolysis”;

“emulsifying agent” refers to an agent that, when added to an emulsion, cause formation of an emulsion and will improve the stability of such emulsion (because emulsion is often thermodynamically unstable);

“stiffening agent” refers to an agent that improves the consistency of an emulsion;

“stabilizer” refers to an agent that improves stability of the emulsion;

“solubilizer” refers to an agent that facilitates the dissolution of solutes into liquids;

“emollient” refers to an agent, when applied locally onto skin, would increase tissue moisture content, thereby rendering the skin softer and more pliable;

“smoothing agent” refers to an agent that improve the after-feel;

“humectant” refers to an agent that incorporates moisture to the skin;

“antifoaming agent” refers to an agent that prevents the foaming of the emulsion when applied onto the skin;

“corticosteroid” encompasses biological active analogs and synthetic analogs;

“polysorbate 80” refers to polyoxyethylene 20 sorbitan monoleate. Polysorbates are a series of partial fatty acid esters of sorbitol and its anhydrides copolymerized with approximately 20, 5 or 4 moles of ethylene oxide for each mole of sorbitol and its anhydrides. Polysorbates may function as an emulsifying agent or solubilizing agent. For purposes of this invention, polysorbate 80 represents an example of polyoxyethylene 20 sorbitan monoleate and encompasses equivalents such as polysorbates 20, 40, 60 and the like.

“Span 60” refers to sorbitan monostearate and is a series of mixtures of partial esters of sorbitol and mono- or di-anhydrides with fatty acids. They may function as an emulsifying agent, or solubilizing agents.

“Microcrystalline wax” refers to a mixture of straight chain and randomly branched saturated alkanes obtained from petroleum. The carbon chain lengths range from C₄₁ to C₅₇; cyclic hydrocarbons are also present. It may act as stiffening agent and can increase the consistency and viscosity of creams and ointments.

“Cetyl palmitate” refers to cetyl ester wax and is a mixture consisting primarily of esters of saturated fatty alcohol (C₁₄-C₁₈) and saturated fatty acids (C₁₄-C₁₈). It may act as an stiffening agent.

“Polawax” is a non-ionic emulsifying wax. It can be prepared from cetostearyl alcohol. It contains a polyoxyethylene derivative of fatty acid ester of sorbitan. Non-ionic emulsifying wax is used as an emulsifying agent in the production of oil-in-water emulsions that are unaffected by moderate concentrations of electrolytes and are stable over a wide range of pH.

“preservative” refers to an agent that inhibits or prevents the growth of microorganisms;

“dry skin condition” encompasses a skin exhibiting, at least, some pathological features of xerosis, hyperkeratosis, parakeratosis, fissuring, cracking, scaling or dryness;

“xerosis” refers to pathologic dryness of the skin (xeroderma);

“xeroderma” is a mild form of ichthyosis characterized by excessive dryness of the skin due to slight increase of the horny layer and diminished water content of the stratum corneum from, for example, decreased perspiration, wind, low humidity, or aging;

“ichthyosis” refers to congenital disorders of keratinization characterized by non-inflammatory dryness and scaling of the skin, often associated with other defects;

“emulsion” refers to a dispersed system containing at least two immiscible liquid phases (e.g., oil and water) that is stabilized with an emulsifying agent to form a single phase; hence, an emulsion in which the oil is dispersed as droplets throughout the aqueous phase is termed an oil-in-water emulsion. When water is the disperse phase and oil is the dispersion medium, the emulsion is of the water-in-oil type;

“aqueous-based emulsion” refers to an emulsion that contains water;

“micronization” refers to a process of pulverizing a larger particle to a smaller particle;

“topical administration” refers to local administration to the skin of the composition and its various embodiments, for example, in the treatment of dry skin and dehydration resulting from skin disease, abrasion, sun, chemicals, wind, cold, fire, renal disease, colonic disease, hemorrhage, vasopressin imbalances, hypothalamic dysfunction, neurohypophyseal dysfunction, or other endocrine abnormalities;

“mammal” refers to a class of higher vertebrates comprising man and all other animals that nourish their young with milk secreted by mammary glands and have the skin usually more or less covered with hair; and

“treating” is intended to encompass relieving, alleviating or eliminating at least one symptom of a dry skin diseases in a mammal.

Unless otherwise specified, % refers to % wt; and % wt refers to % of weight of respective component with respect to the total weight of the composition. For the purposes of the present invention, the term “about” refers to ±10% of a specified value.

The present inventors have unexpectedly discovered a stable aqueous-based emulsion formulation comprising urea and salicylic acid and a method for treating a patient for a skin condition, comprising the step of administering such a stable aqueous-based emulsion formulation. The present composition and method offer significant advantages over the prior art in that it provides enhanced stability of a composition that is useful in treating the skin conditions.

The present invention provides an improved aqueous-based emulsion formulation for urea and salicylic acid exhibiting both non-greasy and stability properties that is an ideal choice for topical treatment of skin conditions. It was the object of the present invention to develop such a stable aqueous-based emulsion formulation comprising a moisturizing agent and a keratolytic agent that are stable.

While urea and salicylic acid have excellent stability in ointment topical dosage forms where an oily base is used, a cream formulation having aqueous base is known to have reduced stability. We surprisingly found a stable, aqueous-based cream formulation having salicylic acid and urea. Both salicylic acid and urea are found to be stable in the present aqueous-based emulsion under the specified storage conditions. The present aqueous-based emulsion is not greasy, in contrast to that of the ointment-based formulation. Without wishing to be bound by a theory, it is believed that the necessity of using water and an unique combination of an emulsifying agent, stiffening agent and/or solubilizer in an aqueous-based cream formulation results in increased stability of urea and salicylic acid, thereby offering a non-greasy alternative formulation.

The present invention is directed to a stable aqueous-based emulsion formulation comprising a moisturizing agent and a keratolytic agent. The present invention is directed to a stable aqueous-based emulsion formulation comprising a moisturizing agent that functions to restore water to the skin. Moisturizing agent includes, but not limited to, urea, glycerin, sorbitol, propylene glycol and the like. Preferably, moisturizing agent is urea. The best time to apply a moisturizer is when the skin is already moistened immediately after a bath or shower, for instance.

The precise percentage of the moisturizing agent in the aqueous-based emulsion formulation may vary depending upon what moisturizing agent is, but it will generally be within the range of from about 8% to about 15% of the formulation. Preferably, the moisturizing agent is between about 9% to about 12%. More preferably, the moisturizing agent is about 10%.

The present invention is directed to a stable aqueous-based emulsion formulation comprising a keratolytic agent that removes the dead and damaged skin cells. Keratolytic agents include, but are not limited to, beta-hydroxy acids such as salicylic acid, benzoyl peroxide and the like. For the purposes of the present invention, salicylic acid encompasses especially alkylated derivatives such as 5-(n-octanoyl) salicylic acid and its esters. Other keratolytic agents include beta-hydroxycarboxylic acids, beta-ketocarboxylic acids, and the like.

The precise percentage of the keratolytic agent in the present aqueous-based emulsion formulation may vary depending upon what the keratolytic agent is, but it will generally be within the range of from about 6% to about 15% of the formulation. Preferably, the keratolytic agent is between about 6% to about 9%. More preferably, the keratolytic agent is about 6%.

In one preferred embodiment, the present invention is directed to a stable aqueous-based emulsion formulation comprising a micronized keratolytic agent. In particular, the present invention provides a stable formulation comprising micronized salicylic acid.

The present invention is directed to a micronized salicylic acid having a mean particle diameter of less than 50μ. Preferably, the micronized salicylic acid has a mean particle diameter of less than 40μ. More preferably, the micronized salicylic acid has a mean particle diameter of less than 25μ. More preferably, the micronized salicylic acid has a mean particle diameter of about 10μ.

Micronization of Salicylic Acid

In one preferred embodiment, the present invention provides a stable formulation comprising micronized salicylic acid and also provides a process for preparing micronized salicylic acid. By the methods of the present invention, salicylic acid, which is prepared by methods known in the art, was first separated by sieves to produce crude salicylic acid having a mean particle diameter of below about 200μ. The sieved crude salicylic acid was then micronized by air jet micronization (a well proven technique that consistently produces particles in the 1-50 micron range). Other micronization methods known in the art, e.g., in a micronizer may be used to obtain micronized salicylic acid. For example, micronization methods include using a ball mill, ultrasonic means, fluid energy attrition mills, a jet mill, or other suitable means as disclosed in Pharmaceutical Dosage Forms: Tablets, Vol. 2, 2^{nd} Ed., Lieberman et al. Ed., Marcel Dekker, Inc. New York (1990) p. 107-200, the content of which is incorporated by reference herein.

Preferably, micronized salicylic acid has a mean particle size of less than about 50μ. Preferably, micronized salicylic acid has a mean particle size of less than about 40μ. Preferably, micronized salicylic acid has a mean particle size of less than about 25μ. Preferably, micronized salicylic acid has a mean particle diameter of less than about 10μ. More preferably, micronized salicylic acid has a mean particle diameter of about 10μ.

The stable aqueous-based emulsion formulation of the present invention may be prepared in the form of cream. Other suitable form includes lotion. Cream and lotion are often similar to one another, differing mainly in their viscosity (creams are typically thicker and more viscous than lotions); both lotion and cream may be opaque, translucent or clear.

The stable aqueous-based emulsion formulation comprises water as a solvent. Preferably, water is from about 15% to about 50%. More preferably, water is from about 20% and 45%. More preferably, water is about 30%.

The stable aqueous-based emulsion formulation comprises an emulsifying agent. In one preferred embodiment, emulsifying agent used in the present invention is a mixture of multiple emulsifiers or a single emulsifier. Suitable emulsifying agent in the present stable formulation includes, but not limited to, a mixture of glyceryl stearate and PEG 100 stearate, glyceryl stearate and PEG 75 stearate, glyceryl stearate and PEG 30 stearate. Preferably, the emulsifying agent is a mixture of glyceryl stearate and PEG 100 stearate.

The wt/wt ratio of glyceryl stearate to PEG 100 stearate, is between about 1:4 to about 4:1. More preferably, The wt/wt ratio of glyceryl stearate to PEG 100 stearate is about 1:1.

The wt/wt ratio of glyceryl stearate to PEG 75 stearate, is between about 1:4 to about 4:1. More preferably, The wt/wt ratio of glyceryl stearate to PEG 75 stearate is about 1:1.

The wt/wt ratio of glyceryl stearate to PEG 30 stearate, is between about 1:4 to about 4:1. More preferably, The wt/wt ratio of glyceryl stearate to PEG 30 stearate is about 1:1.

In another preferred embodiment, other suitable emulsifying agent may include a mixture of glyceryl stearate, PEG 100 stearate, tribehenin, cetearyl alcohol, PEG-20 cetyl phophate, diacetyl phosphate and cetyl palmitate.

Preferably, the emulsifying agent is present in the stable formulation from about 2% to about 25%. More preferably, the emulsifying agent is present in the stable formulation from about 5% to about 20%. More preferably, the emulsifying agent is present in the stable formulation of about 15%.

In another yet preferred embodiment, the emulsifying agent present in the stable aqueous-based emulsion formulation may be polawax, polysorbate 80 or Span 60. The emulsifying agent may be a mixture of emulsifying agents or a single emulsifying agent. More preferably, the emulsifying agent is polawax. Addition of polysorbate 80 or Span 60 is optional. The emulsifying agent may contain a mixture of polawax, polysorbate 80 and Span 60.

We surprisingly discovered that the use of the present emulsifying agents contributes to an aqueous-base formulation containing urea and salicylic acid that is non-greasy when applied to skin and exhibits good stability when storage.

The stable aqueous-based emulsion formulation comprises a stiffening agent. Suitable stiffening agent in the present stable formulation includes, but not limited to, cetyl alcohol, stearyl alcohol, microcrystalline wax, cetyl palmitate, and the like.

Preferably, the stiffening agent is from about 0.1% to about 10%. More preferably, the stiffening agent is from about 0.5% to about 5%. More preferably, the stiffening is about 1.0%.

The stable aqueous-based emulsion formulation may comprise a solubilizer. Exemplary solubilizer includes propylene glycol, hexylene glycol, polysorbate 80, di-PPG-myreth-10-adipate, and the like.

Preferably, the solubilizer is from about 10% to 30%. More preferably, the solubilizer is about 15%.

The stable aqueous-based emulsion formulation comprises a stabilizer. Suitable stabilizer in the present stable formulation includes, but not limited to, xanthan gum and the like.

Preferably, the stabilizer is from about 0.001% to about 3%. More preferably, the stabilizer is from about 0.01% to about 1%. More preferably, the stabilizer is about 0.1%.

The stable aqueous-based emulsion formulation may optionally comprise an emollient, additional emulsifying agent, smoothing agent (imparts silky feel), humectants, antifoaming agent, and preservative.

The stable aqueous-based emulsion formulation may further comprise an oleaginous vehicle. Exemplary oleaginous vehicle is soybean oil, mineral oil and the like.

The stable aqueous-based emulsion formulation may further contain sodium hydroxide to adjust the pH of the formulation. Other suitable pH adjusters that are known in the art may be used. Preferably, the formulation may have a pH of about 4-6. More preferably, the formulation has a pH of about 4-4.5.

Suitable emollient includes, but not limited to, mineral oil light. Additional emollients include mineral oil, almond oil, castor oil, soybean oil, cottonseed oil, mineral oil and lanolin alcohols, isopropyl myristate, isopropyl palmitate and sunflower oil. Suitable additional emulsifying agents include, but not limited to, stearic acid and polysorbate 80. Additional emulsifying agents include polysorbate 20, polysorbate 40, polysorbate 60 and the like. Suitable smoothing agent (imparts silky feel) includes, but not limited to, cyclomethicone and dimethicone. Suitable humectant includes, but not limited to, glycerin and propylene glycol. Additional humectants include sorbitol, ethylene glycol and xylitol. Suitable antifoaming agent includes, but not limited to, simethicone emulsion and the like. Suitable preservative includes, but not limited to, benzyl alcohol or propylene glycol and the like. Additional preservatives include methylparaben, propylparaben, ethylparaben sorbic acid, benzoic acid, sodium benzoate, dichlorobenzyl alcohol, formaldehyde and the like.

The stable aqueous-based emulsion formulation may optionally further comprise a fragrance and dye/colorant. Suitable fragrances and colors, such as FD&C Red No. 40 and FD&C Yellow No. 5, may be used in the stable formulations of the present invention. Other examples of fragrances and colors suitable for use in topical formulation are well known in the art.

In one preferred embodiment, the present stable aqueous-based emulsion formulation may include an anti-fungal agent. The novel formulation of the present invention provides adequate penetration of anti-fungal agents for treating superficial fungal infections. Exemplary anti-fungal agent includes, but not limited to, amphoteracin B, clotrimazole, econazole, micronazole, terconazole, butoconazole, tioconazole, oxiconazole, sulconazole, ciclopirox olamine, haloprogin, tolnaftate, naftifine, terbinafine, nystatin, amophotericin B and the like. Miscellaneous antifungal agents such as undecylenic acid, benzoic acid and salicylic acid, propionic acid and caprylic acid, potassium iodide and the like. More preferably, the anti-fungal agent is terbinafine. For purposes of the present invention, the term “terbinafine” is intended to include both the free base form and its corresponding salt form (e.g., hydrochloride). The present formulation may include one or more of the anti-fungal agents.

The amount of anti-fungal agent is present at a therapeutic amount to treat fungal infection. Preferably, the anti-fungal agent is present in the amount of about 0.1% to about 10%. More preferably, the anti-fungal agent is present in the amount of about 1% to about 5%. More preferably, the anti-fungal agent is present at about 3% or at about 5%.

In another preferred embodiment, the present stable aqueous-based emulsion formulation may include a corticosteroid. Exemplary corticosteroid includes, but not limited to, alclometasone dipropionate, amcinonide, beclomethasone dipropionate, betamethasone, betamethasone benzoate, betamethasone dipropionate, desonide, desoximetasone, betamethasone valerate, clobetasol propionate, clocortolone pivalate, cortisol, cortisone, fludrocortisone, flunisolide, fluocinonide, prednisone, prednisolone, 6α-methylprednisolone, triamcinolone, dexamethasone, mometasone furoate and the like. More preferably, corticosteroid is desoximetasone. The present formulation may include one or more corticosteroids.

The amount of corticosteroid is present at a therapeutic amount to treat inflammatory diseases. Preferably, the corticosteroid is present in the amount of about 0.01% to about 5%. More preferably, the corticosteroid is present in the amount of about 0.1% to about 2%. More preferably, the corticosteroid is present at about 1%.

In another preferred embodiment, the present stable aqueous-based emulsion formulation may include a macrolide lactone as exemplified by tacrolimus, pimecrolimus, and the like. The amount of macrolide lactone is present at a therapeutic amount to treat bacterial infection.

In another preferred embodiment, the present stable aqueous-based emulsion formulation may include a photoprotectant or antioxidant such as genestein, vitamin C, vitamin E, avobenzone, octinoxate, and the like. Photoprotectant is an agent that prevents UV penetration into skin. The amount of photoprotectant or antioxidant is present at a therapeutic amount to render photoprotection and antioxidation of skins.

In another preferred embodiment, the present stable aqueous-based emulsion formulation may include an anesthetic, such as lidocaine, pramoxine, and the like. The amount of anesthetic is present at a therapeutic amount to cause anesthetic effects.

In another preferred embodiment, the present stable aqueous-based formulation may include a vitamin D₃ derivative such as calcipotriene. Calcipotriene is known for its anti-psoriasis activity. The amount of vitamin D₃ derivative is present at a therapeutic amount to treat psoriasis.

In another preferred embodiment, the present stable aqueous-based formulation may include an anti-viral or anti-neoplastic agent such as 5-fluorouracil, imiqumoid or cidofovir. The amount of anti-viral or anti-neoplastic agent is present at a therapeutic amount to treat viral or neoplastic diseases.

The present invention provides a method of preparing a stable aqueous-based emulsion formulation. The preparation method is detailed hereinafter.

Methods of Preparation

In one preferred embodiment, the present invention provides a method for preparing a stable aqueous-based formulation, comprising the steps of:
a) combining an emulsifying agent with a stiffening agent to form a first solution;
b) dissolving a moisturizing agent in water to form a second solution;
c) adding the second solution to the first solution to form an emulsion; and
d) adding a keratolytic agent to the emulsion to form a stable aqueous-based formulation.

In another preferred embodiment, the present invention further provides a method for preparing a stable aqueous-based formulation, comprising the steps of:
a) preparing a first solution of emulsifying agent;
b) dissolving a moisturizing agent in water to form a second solution;
c) adding the second solution to the first solution to form an emulsion;
d) adding a keratolytic agent to a solubilizer to form a third solution; and
e) adding the third solution to the emulsion to form a stable aqueous-based formulation.

Specifically, the details of the preparative process are provided as below:

Step i): The oil phase components, which include glyceryl stearate and PEG 100 stearate, cetyl alcohol, stearyl alcohol, mineral oil light and stearic acid were heated to 70±3° C. in a stainless steel prep kettle;

Step, ii): The water phase components, which include purified water was taken in a Brogli mixing vessel and heated to 68±3° C. Urea and glycerin were added and mixed. Xanthan gum was slowly added with the homogenizer speed at 2,000 RPM and with a gap setting of 2.5-3.0. To the above solution, benzyl alcohol, propylene glycol, polysorbate 80 and simethicone emulsion were added. The water phase components were mixed at 39 RPM and the temperature was maintained at 68±3° C.;

Step, iii): Under vacuum (−400±200 mbar) and under control transfer rate (through a 3/16th diameter hole) the oil phase was incorporated to the water phase while mixing at 39 RPM and maintaining the temperature at 65±3° C. The emulsion was mixed for 10 minutes after the completion of transfer;

Step iv): Under vacuum (−600±200 mbar) and while mixing at 39 RPM the emulsion was cooled to 25±2° C. The emulsion was homogenized at 3,000 RPM with a gap setting of 1.0 when it reached 50° C. and was stopped at 45° C.;

Step v): Cyclomethicone was added to the above batch and mixed at 25±2° C.;

Step vi): Salicylic acid was added while mixing at 39 RPM to the above batch with the homogenizer gap set of 3.0 and with a homogenizing speed of 3,000 RPM. The homogenizing and mixing was performed for 60±10 minutes.

The present invention is directed to a method of treating skin conditions in a mammal. More particularly, the present invention provides a method of treating skin conditions in a patient by providing an improved stable formulation comprising therapeutic effective amounts of urea and salicylic acid. According to the present invention, the term “patient” will encompass any mammal requiring treatment for skin conditions, particularly a human patient suffering from dry skin condition.

The present aqueous-based emulsion formulation can provide both moisturizing and keratolytic effects on several dry skin conditions. Dry skin conditions include, but not limited, ichthyosis. Ichthyosis has a symptom ranging from mild but annoying dryness to severe dryness with scales and flaking that becomes disfiguring. Ichthyotic skin diseases may be inherited. Inherited ichthyoses are characterized by excessive accumulation of scale on the skin surface and are classified according to clinical and genetic criteria. For example, ichthyosis is a symptom in Refsum's syndrome (rare hereditary ataxia with polyneuritic changes and deafness caused by a defect in the enzyme phytanic acid hydroxylase) and in Sjögren-Larsson syndrome (hereditary mental deficiency and spastic paralysis). Both the Refsum's and the Sjögren-Larsson syndromes are autosomal recessive. Acquired ichthyosis may be an early manifestation in some systemic diseases (e.g., leprosy, hypothyroidism, lymphoma, AIDS). The dry scaling may be fine and localized to the trunk and legs, or it may be thick and widespread. Biopsy of ichthyotic skin is usually not diagnostic; however, there are exceptions, most notably sarcoidosis, in which a thick scaling may appear on the legs and biopsy usually shows typical granulomas.

Xeroderma (xerosis) represents the mildest form of ichthyosis. Xerodmera is neither congenital nor associated with systemic abnormalities. It usually occurs on the lower legs of middle-aged or older patients, most often during cold weather and in those who bathe too frequently. There may be mild to moderate itching and an associated dermatitis caused by detergents or other irritants.

For the purpose of the present invention, dry skin conditions include, but are not limited to, mild to moderate xerosis, as an exfoliant of the dead cells. The treatment of xerosis has three aspects: replacing water content and maintaining hydration, alleviating the symptomatology, and normalizing the degree of keratinization which generally reduces scaling.

The dosage for the urea and salicylic acid compositions of the invention will vary depending on several factors, including, but not limited to, the age, weight, and species of the patient, the general health of the patient, the severity of the symptoms in skin conditions.

Analytical Protocols:

The stability of urea and salicylic acid in the aqueous-based emulsion formulation was determined by High Performance Liquid Chromatography (HPLC).

Content of urea was determined using the following protocol. In this protocol, urea content was determined by a HPLC instrument with a chromatograph and an ultra-violet detector. The column used was a Waters Atlantis™ Hydrophilic Interaction Chromatography (HILIC) silica, 250×4.6 mm, 5 μm. HILIC is a variation of normal-phase chromatography without the disadvantages of using solvents that are not miscible with water. In addition, a guard column like the Waters Atlantis™ HILIC Silica, 20×4.6 mm, 5 μm was used. The detection was performed using an ultra-violet wavelength at 195 nanometer. The mobile phase consisted of acetonitrile (96%) and Water (4%). The flow rate was set at 2.0 mL/min with an injection volume of 10 μL and with a run time of about 10 minutes.

Content of salicyclic acid was determined using the following protocol. In this protocol, a HPLC instrument with a chromatograph and an ultra-violet detector was used. The column used was an Agilent Zorbax Eclipse Extra Density Bonded (XDB-C18), 250×4.6 mm, 5 μm. ZORBAX columns was built from porous silica microspheres (PSM) based on silica sols. In addition, a suitable guard column like the Phenomenex® ODS 4×3.0 mm was used. The detection was performed using an ultra-violet wavelength at 215 nm. The mobile phase consisted of 0.085% phosphoric acid in water. The flow rate was set at 1.0 mL/min with an injection volume of 10 μL and with a run time of about 37 minutes.

Content of terbinafine was determined using the following protocol. In this protocol, is HPLC instrument with a chromatograph and ultraviolet detector was used. The column used was an Agilent Zorbax SB Phenyl 250×4.6 mm, 5 μm. The detection was performed using ultraviolet light at 224 nm. Mobile phase A was 50 mM ammonium phosphate buffer pH 3.0 and mobile phase B was acetonitrile. The column was eluted with a gradient from 20% A, 80% B to 50% A, 50% B over 30 minutes. The flow rate was set at 1.3 mL/min with an injection volume of 10 μL and run time of 40 minutes. The column temperature was set at 40° C.

Having now generally described this invention, the same will be better understood by reference to the following Examples, which are provided herein solely for purposes of illustration only and are not intended to be limiting of the invention or any embodiment thereof, unless so specified. All parts and percentages referred to in this specification and the appended claims are by weight unless otherwise specified.

### EXAMPLES

### Example 1

The following table lists an exemplary stable emulsion formulation containing urea and salicylic acid. An example of this invention prepared as an aqueous-based emulsion is described in the following formulation in Table 1.

TABLE 1
A Stable Aqueous-Based Emulsion Formulation No. 1

| | Weight (500 | | % |
|---|---|---|---|
| Ingredients | grams) | | Total |
| Moisturizing Agent | | | |
| Urea | 50.0 | grams | 10.0 |
| Keratolytic Agent | | | |
| Salicylic Acid | 50.0 | grams | 10.0 |
| Solvent | | | |
| Purified water | 222.0 | grams | 44.4 |
| Emulsifying Agent | | | |
| Glyceryl Stearate and PEG 100 Stearate | 85.0 | grams | 17.0 |
| Stiffening Agent | | | |
| Cetyl alcohol | 5.0 | grams | 1.0 |
| Stearyl alcohol | 5.0 | grams | 1.0 |
| Solubilizer | 0 | gram | 0.0 |
| Other Excipients | | | |
| Xanthan gum (stabilizer) | 0.5 | grams | 0.1 |
| Mineral oil light (emollient) | 15.0 | grams | 3.0 |
| Stearic acid (additional emulsifying agent) | 5.0 | grams | 1.0 |
| Polysorbate 80 (additional emulsifying agent) | 10.0 | grams | 2.0 |
| Cyclomethicone (smoothing agent) | 2.5 | grams | 0.5 |
| Glycerin (humectant) | 10.0 | grams | 2.0 |
| Propylene glycol (humectant) | 25.0 | grams | 5.0 |
| Simethicone (antifoaming agent) | 10.0 | grams | 2.0 |
| Benzyl alcohol (preservative) | 5.0 | grams | 1.0 |

The stable aqueous-based emulsion formulation composition as disclosed in this example was prepared in the following manner. In a stainless steel prep kettle, the following oil phase components (i.e., glyceryl stearate and PEG 100 stearate, cetyl alcohol, stearyl alcohol, mineral oil light and stearic acid were heated to 70±3° C. The water phase components (i.e., purified water) was taken in a Brogli mixing vessel and heated to 68±3° C. Urea and glycerin were then added and mixed. Xanthan gum was slowly added with the homogenizer ON at 2,000 RPM and with a gap setting of 2.5-3.0. Benzyl alcohol, propylene glycol, polysorbate 80 and simethicone emulsion were added to the water phase. The water phase components were mixed at 39 RPM and the temperature was maintained at 68±3° C.

The oil phase was incorporated to the water phase under vacuum (−400±200 mbar) and under control transfer rate (through a 3/16th diameter hole) while mixing at 39 RPM and maintaining the temperature at 65±3° C. The emulsion was mixed for 10 minutes after the completion of transfer. Under vacuum (−600±200 mbar) and while mixing at 39 RPM the emulsion was cooled to 25±2° C. The emulsion was homogenized at 3,000 RPM with a gap setting of 1.0 when it reached 50° C. and was stopped at 45° C. Cyclomethicone was added to the above batch and mixed. Salicylic acid was added while mixing at 39 RPM to the above batch with the homogenizer gap set of 3.0 and a homogenizing speed of 3,000 RPM. The homogenizing and mixing was performed for 60±10 minutes.

### Example 2

The following table lists an exemplary stable emulsion formulation containing urea and salicylic acid.

TABLE 2
A Stable Aqueous-Based Emulsion Formulation No. 2

| | Weight (500 | | % |
|---|---|---|---|
| Ingredients | grams) | | Total |
| Moisturizing Agent | | | |
| Urea | 50.0 | grams | 10.0 |
| Keratolytic Agent | | | |
| Salicylic Acid | 50.0 | grams | 10.0 |
| Solvent | | | |
| Purified water | 114.5 | grams | 22.9 |
| Emulsifying Agent | | | |
| Glyceryl Stearate and PEG 100 Stearate | 60.0 | grams | 12.0 |
| Tribehenin | 10.0 | grams | 2.0 |
| Cetearyl Alcohol (and) PEG-20 cetyl | 25.0 | grams | 5.0 |
| phosphate (and) diacetyl phosphate | | | |
| Cetyl Palmitate | 25.0 | grams | 5.0 |
| Stiffening Agent | 0 | gram | 0.0 |
| Solubilizer | | | |
| Hexylene Glycol | 150.0 | grams | 30.0 |
| Other Excipients | | | |
| Xanthan gum (stabilizer) | 0.5 | grams | 0.1 |
| (emollient) | 0.0 | grams | 0.0 |
| Stearic acid (additional emulsifying agent) | 10.0 | grams | 2.0 |
| (smoothing agent) | 0 | gram | 0.0 |
| (humectant) | 0 | gram | 0.0 |
| Simethicone (antifoaming agent) | 2.5 | grams | 0.5 |
| Benzyl alcohol (preservative) | 2.5 | grams | 0.5 |

The stable aqueous-based emulsion formulation composition as disclosed in this example was prepared in the following manner. The oil phase components that include glyceryl stearate and PEG 100 stearate, tribehenin, cetyl palmitate, cetearyl alcohol (and) PEG-20 cetyl phosphate (and) diacetyl phosphate and stearic acid were heated to 70° C. The salicylic acid was added to hexylene glycol and dissolved. The dissolved portion was added to the oil portion. The xanthan gum was added to the purified water, simethicone and benzyl alcohol mixed and heated to 70° C. The oil phase ingredients were added to the water phase ingredients mixed and homogenized. The emulsion was cooled to 30° C.

### Example 3

The following table lists an exemplary stable emulsion formulation containing urea and salicylic acid.

TABLE 3
A Stable Aqueous-Based Emulsion Formulation No. 3

| | Weight (500 | | % |
|---|---|---|---|
| Ingredients | grams) | | Total |
| Moisturizing Agent | | | |
| Urea | 50.0 | grams | 10.0 |
| Keratolytic Agent | | | |
| Salicylic Acid | 50.0 | grams | 10.0 |
| Solvent | | | |
| Purified water | 122.0 | grams | 24.4 |
| Emulsifying Agent | | | |
| Glyceryl Stearate and PEG 100 Stearate | 40.0 | grams | 8.0 |
| Tribehenin | 15.0 | grams | 3.0 |
| Cetearyl Alcohol (and) PEG-20 cetyl | 30.0 | grams | 6.0 |
| phosphate (and) diacetyl phosphate | | | |
| Cetyl Palmitate | 30.0 | grams | 6.0 |
| Stiffening Agent | 0 | gram | 0.0 |
| Solubilizer | | | |
| Hexylene Glycol | 75.0 | grams | 15.0 |
| Di-PPG2-Myreth-10-Adipate | 75.0 | grams | 15.0 |
| Other Excipients | | | |
| Xanthan gum (stabilizer) | 0.5 | grams | 0.1 |
| (emollient) | 0 | gram | 0.0 |
| Stearic acid (additional emulsifying agent) | 10.0 | grams | 2.0 |
| (smoothing agent) | 0 | gram | 0.0 |
| (humectant) | 0 | gram | 0.0 |
| (antifoaming agent) | 0 | gram | 0.0 |
| Benzyl alcohol (preservative) | 2.5 | grams | 0.5 |

The stable aqueous-based emulsion formulation composition as disclosed in this example was prepared in the following manner. The oil phase components that include glyceryl stearate and PEG 100 stearate, tribehenin, cetyl palmitate, cetearyl alcohol (and) PEG-20 cetyl phosphate (and) diacetyl phosphate and stearic acid were heated to 70° C., The salicylic acid was added to a mixture of hexylene glycol and Di-PPG2-myreth-10-adipate and dissolved. The dissolved portion was added to the oil portion. The xanthan gum was added to the Purified water and Benzyl alcohol mixed and heated to 70° C. The oil phase ingredients were added to the water phase ingredients mixed and homogenized. The emulsion was cooled to 30° C.

### Example 4

The following table lists an exemplary stable emulsion formulation containing urea and salicylic acid.

TABLE 4
A Stable Aqueous-Based Emulsion Formulation No. 4

| | Weight (500 | | % |
|---|---|---|---|
| Ingredients | grams) | | Total |
| Moisturizing Agent | | | |
| Urea | 50.0 | grams | 10.0 |
| Keratolytic Agent | | | |
| Salicylic Acid | 50.0 | grams | 10.0 |
| Solvent | | | |
| Purified water | 114.5 | grams | 22.9 |
| Emulsifying Agent | | | |
| Glyceryl Stearate and PEG 100 Stearate | 60.0 | grams | 12.0 |
| Tribehenin | 10.0 | grams | 2.0 |
| Cetearyl Alcohol (and) PEG-20 cetyl | 25.0 | grams | 5.0 |
| phosphate (and) diacetyl phosphate | | | |
| Cetyl Palmitate | 25.0 | grams | 5.0 |
| Stiffening Agent | 0 | gram | 0.0 |
| Solubilizer | | | |
| Hexylene Glycol | 75.0 | grams | 15.0 |
| Polysorbate 80 | 75.0 | grams | 15.0 |
| Other Excipients | | | |
| Xanthan gum (stabilizer) | 0.5 | gram | 0.1 |
| (emollient) | 0 | gram | 0.0 |
| Stearic acid (additional emulsifying agent) | 10.0 | grams | 2.0 |
| (smoothing agent) | 0 | gram | 0.0 |
| (humectant) | 0 | gram | 0.0 |
| Simethicone (antifoaming agent) | 2.5 | grams | 0.5 |
| Benzyl alcohol (preservative) | 2.5 | grams | 0.5 |

The stable aqueous-based emulsion formulation composition as disclosed in this example was prepared in the following manner. The oil phase components that include glyceryl stearate and PEG 100 stearate, tribehenin, cetyl palmitate, cetearyl alcohol (and) PEG-20 cetyl phosphate (and) diacetyl phosphate and stearic acid were heated to 70° C. The salicylic acid was added to a mixture of hexylene glycol and Polysorbate 80 and dissolved. The dissolved portion was added to the oil portion. The xanthan gum was added to the Purified water, simethicone and Benzyl alcohol mixed and heated to 70° C. The oil phase ingredients were added to the water phase ingredients mixed and homogenized. The emulsion was cooled to 30° C.

### Example 5

The following table summarizes the stability of the stable aqueous-based emulsion formulation as prepared in Example 1 under the accelerated storage condition of 35° C. The urea, salicylic acid and benzyl alcohol contents were normalized based on the original amounts added (during the time when formulation was prepared) at 0 week time point test result.

TABLE 5
Stability Studies of Aqueous Based Emulsion Formulation No. 1

| Test | | | | |
|---|---|---|---|---|
| Parameters | 0 week | 2 weeks | 4 weeks | 6 weeks |
| Urea content | 100.0% | 100.8% | 97.2% | 98.6% |
| Salicylic Acid | 100.0% | 98.7% | 98.2% | 98.3% |
| content | | | | |
| Benzyl Alcohol | 100.0% | 99.4% | 97.7% | 96.4% |
| content | | | | |

The content of urea, salicylic acid and benzyl alcohol did not decrease significantly even after 6 week-storage under accelerated condition.

### Example 6

The following table summarizes additional consistent assay results of salicylic acid urea and benzyl alcohol of the cream formulation as prepared in Example 1. The stability was conducted at 35° C. and 60% relative humidity (RH). The stable aqueous-based emulsion formulation was prepared in a commercial scale-manufacturing kettle.

TABLE 6
Stability Studies of Aqueous Based Emulsion Formulation No. 1

| | | Salicylic acid | Benzyl Alcohol |
|---|---|---|---|
| Experiments | Urea content | content | content |
| 0 week | 102.4% | 101.9% | 96.1% |
| 4 weeks | 100.0% | 103.2% | 96.4% |
| 0 week | 102.2% | 101.8% | 98.8% |
| 4 weeks | 102.5% | 102.4% | 98.3% |
| 0 week | 101.9% | 100.8% | 99.0% |
| 4 weeks | 100.3% | 101.8% | 95.3% |

The stability date for urea, salicylic acid and benzyl alcohol were found to be consistent in three separate set of experiments.

### Example 7

The following table summarizes the stability of the cream formulation as prepared in Example 1, 2, 3 and 4 under the accelerated stability condition of 35° C. and 40° C.

TABLE 7
Physical Characteristics of Various Aqueous Based Emulsion Formulations

| | Storage Condition | | | |
|---|---|---|---|---|
| | Physical | Physical | Physical | |
| | Characteristics | Characteristics | Characteristics | Physical Characteristics |
| | T = 35° C. | T = 40° C. | T = 35° C. | T = 40° C. |
| Formulations | 1 week | 1 week | 2 weeks | 2 weeks |
| Formulation | Physically | Physically | Physically | Physically |
| #1 | stable. Smooth | stable. Smooth | stable. Smooth | stable. Smooth |
| | emulsion with | emulsion with | emulsion with | emulsion with |
| | characteristic | characteristic | characteristic | characteristic |
| | odor. | odor. | odor. | odor. |
| Formulation | Physically | Physically | Physically | Physically |
| #2 | stable. Smooth | stable. Smooth | stable. Smooth | stable. Smooth |
| | emulsion with | emulsion with | emulsion with | emulsion with |
| | characteristic | characteristic | characteristic | characteristic |
| | odor. | odor. | odor. | odor. |
| Formulation | Physically | Physically | Physically | Physically |
| #3 | stable. Smooth | stable. Smooth | stable. Smooth | stable. Smooth |
| | emulsion with | emulsion with | emulsion with | emulsion with |
| | characteristic | characteristic | characteristic | characteristic |
| | odor. | odor. | odor. | odor. |
| Formulation | Physically | Physically | Physically | Physically |
| #4 | stable. Smooth | stable. Smooth | stable. Smooth | stable. Smooth |
| | emulsion with | emulsion with | emulsion with | emulsion with |
| | characteristic | characteristic | characteristic | characteristic |
| | odor. | odor. | odor. | odor. |

T = Temperature

### Example 8

The following table lists a further exemplary stable emulsion formulation containing urea and salicylic acid. In this series of study, the prepared formulation nos. 5-7 contain emulsifying agents such as polawax or polysorbate 80 and stiffening agents such as cetyl palmitate or microcrystalline wax. An example of this invention prepared as an aqueous-based emulsion is described in the following formulation in Table 8.

TABLE 8
A Stable Aqueous-Based Emulsion Formulation No. 5

| | Weight (300 | | % |
|---|---|---|---|
| Ingredients | grams) | | Total |
| Moisturizing Agent | | | |
| Urea | 30.0 | grams | 10.0 |
| Keratolytic Agent | | | |
| Salicylic Acid | 16.5 | grams | 5.5 |
| Solvent | | | |
| Purified water | 101.85 | grams | 33.95 |
| Emulsifying Agent | | | |
| Polawax | 45.0 | grams | 15.0 |
| Polysorbate 80 (optional) | 15.0 | grams | 5.0 |
| Span 60 (optional) | 15.0 | grams | 5.0 |
| Stiffening Agent | | | |
| Cetyl palmitate | 6.0 | grams | 2.0 |
| Microcrystalline wax (optional) | 6.0 | grams | 2.0 |
| Solubilizer | | | |
| Propylene glycol | 45 | gram | 15.0 |
| Other Excipients | | | |
| Oleaginous vehicle (soybean oil) | 16.5 | grams | 5.5 |
| Sodium hydroxide (adjusted pH 4-4.5) | 3.15 | grams | 10.5 |

The stable aqueous-based emulsion formulation composition as disclosed in this example was prepared in the following manner. A suitable amount of salicylic acid (5.5%)+was weighed and dissolved in propylene glycol. The mixture was heated at 60° C.

An oil phase was prepared by weighing proper amounts of emulsifying agents (such as polawax, polysorbate 80 (optional) or Span 60 (optional)), stiffening agents (such as cetyl palmitate or microcrystalline wax (optional) as well as soybean oil. They were added together in a container to make up the oil phase and heated to 70° C. The prepared salicylic acid in propylene glycol was then added into the prepared oil phase followed by stirring in order to obtain a homogenous mixture.

A suitable amount of urea and sodium hydroxide were weighed and added into distilled water and heat to 70° C. The prepared urea and sodium hydroxide solution was added to the homogenous oil phase. The mixture was further stirred at 70° C. to make an emulsion (the emulsion may be homogenized at this time). The temperature of the emulsion (i.e., at approximately 70° C. was maintained for a short period of time (i.e., 10 minutes). The emulsion was allowed to cool to room temperature while stirring to form a stable aqueous-based emulsion formulation.

### Example 9

Using the method detailed in Example 8, we prepared formulation no. 6, which represents another exemplary stable emulsion formulation containing urea (10%) and salicylic acid (6%).

TABLE 9
A Stable Aqueous-Based Emulsion Formulation No. 6

| | Weight (300 | | % |
|---|---|---|---|
| Ingredients | grams) | | Total |
| Moisturizing Agent | | | |
| Urea | 30.0 | grams | 10.0 |
| Keratolytic Agent | | | |
| Salicylic Acid | 18.0 | grams | 6.0 |
| Solvent | | | |
| Purified water | 100.35 | grams | 33.45 |
| Emulsifying Agent | | | |
| Polawax | 45.0 | grams | 15.0 |
| Polysorbate 80 (optional) | 15.0 | grams | 5.0 |
| Span 60 (optional) | 15.0 | grams | 5.0 |
| Stiffening Agent | | | |
| Cetyl palmitate | 6.0 | grams | 2.0 |
| Microcrystalline wax (optional) | 6.0 | grams | 2.0 |
| Solubilizer | | | |
| Propylene glycol | 45 | gram | 15.0 |
| Other Excipients | | | |
| Oleaginous vehicle (soybean oil) | 16.5 | grams | 5.5 |
| Sodium hydroxide (adjusted pH 4-4.5) | 3.15 | grams | 10.5 |

### Example 10

Using the method detailed in Example 8, we prepared formulation no. 7, which represents another exemplary stable emulsion formulation containing urea (10%), salicylic acid (6%) and terbinafine hydrochloride (5%).

TABLE 10
A Stable Aqueous-Based Emulsion Formulation No. 7

| | Weight (1,000 | | % |
|---|---|---|---|
| Ingredients | grams) | | Total |
| Moisturizing Agent | | | |
| Urea | 100.0 | grams | 10.0 |
| Keratolytic Agent | | | |
| Salicylic Acid | 60.0 | grams | 6.0 |
| Antifungal agent | | | |
| Terbinafine hydrochloride | 50.0 | grams | 5.0 |
| Solvent | | | |
| Purified water | 234.5 | grams | 23.45 |
| Emulsifying Agent | | | |
| Polawax | 150.0 | grams | 15.0 |
| Polysorbate 80 (optional) | 50.0 | grams | 5.0 |
| Span 60 (optional) | 50.0 | grams | 5.0 |
| Stiffening Agent | | | |
| Cetyl palmitate | 20.0 | grams | 2.0 |
| Microcrystalline wax (optional) | 20.0 | grams | 2.0 |
| Solubilizer | | | |
| Propylene glycol | 200 | gram | 15.0 |
| Other Excipients | | | |
| Oleaginous vehicle (soybean oil) | 55 | grams | 5.5 |
| Sodium hydroxide (adjusted pH 4-4.5) | 10.05 | grams | 10.5 |

### Example 11

The following tables summarizes the stability of the stable aqueous-based emulsion formulation as prepared in Examples 8-10 under the accelerated storage condition of 35° C. or 40° C. and 75% relative humidity (RH). The urea, salicylic acid and terbinafine hydrochloride contents were normalized based on the original amounts added (during the time when formulation was prepared) at 0 week time point test result.

TABLE 11
Stability Studies of Aqueous Based Emulsion
Formulation No. 5 at 35° C.

| Test | | | |
|---|---|---|---|
| Parameters | 0 week | 2 weeks | 4 weeks |
| Urea content | 105.4% | 106.4% | 103.2% |
| Salicylic Acid | 104.9% | 106.9% | 105.7% |
| content | | | |

TABLE 12
Stability Studies of Aqueous Based Emulsion
Formulation No. 5 at 40° C./75%
Relative Humidity

| Test | | | |
|---|---|---|---|
| Parameters | 0 week | 2 weeks | 4 weeks |
| Urea content | 105.4% | 103.5% | 100.9% |
| Salicylic Acid | 104.9% | 104.1% | 104.6% |
| content | | | |

The data show that the prepared emulsion formulation (formulation no. 5) containing 5.5% salicylic acid and 10% urea is stable. The content of urea, and salicylic acid did not decrease significantly after 4 week-storage under accelerated conditions of either 35° C. or 40° C./75% relative humidity.

TABLE 13
Stability Studies of Aqueous Based Emulsion
Formulation No. 6 at 35° C.

| Test | | | |
|---|---|---|---|
| Parameters | 0 week | 2 weeks | 4 weeks |
| Urea content | 105.7% | 107.3% | 102.7% |
| Salicylic Acid | 105.2% | 105.4% | 106.5% |
| content | | | |

TABLE 14
Stability Studies of Aqueous Based Emulsion
Formulation No. 6 at 40° C./75%
Relative Humidity

| Test | | | |
|---|---|---|---|
| Parameters | 0 week | 2 weeks | 4 weeks |
| Urea content | 105.7% | 107.0% | 100.6% |
| Salicylic Acid | 105.2% | 105.2% | 104.1% |
| content | | | |

The data show that the prepared emulsion formulation (formulation no. 6) containing 6% salicylic acid and 10% urea is stable. The content of urea, and salicylic acid did not decrease significantly after 4 week-storage under accelerated conditions of either 35° C. or 40° C./75% relative humidity.

### Example 12

The following table summarizes the stability of the cream formulation as prepared in Example 5, 6, and 7 under the accelerated stability condition of 35° C. and 40° C.

TABLE 15
Physical Characteristics of Various Aqueous Based Emulsion Formulations

| | Storage Condition | | | |
|---|---|---|---|---|
| | | Physical | | Physical |
| | Physical | Characteristics | Physical | Characteristics |
| | Characteristics | T = 40° C./75% | Characteristics | T = 40° C./75% |
| | T = 35° C. | RH | T = 35° C. | RH |
| Formulations | 1 week | 1 week | 2 weeks | 2 weeks |
| Formulation #5 | Physically | Physically | Physically | Physically |
| | stable. Smooth | stable. Smooth | stable. Smooth | stable. Smooth |
| | emulsion with | emulsion with | emulsion with | emulsion with |
| | characteristic | characteristic | characteristic | characteristic |
| | waxy odor. | waxy odor. | waxy odor. | waxy odor. |
| Formulation | Physically | Physically | Physically | Physically |
| #6 | stable. Smooth | stable. Smooth | stable. Smooth | stable. Smooth |
| | emulsion with | emulsion with | emulsion with | emulsion with |
| | characteristic | characteristic | characteristic | characteristic |
| | waxy odor. | waxy odor. | waxy odor. | waxy odor. |
| Formulation | N/A | Physically | N/A | Physically |
| #7 | | stable. Smooth | | stable. Smooth |
| | | emulsion with | | emulsion with |
| | | characteristic | | characteristic |
| | | waxy odor. | | waxy odor. |

T = Temperature;
RH = relative humidity;
N/A = studies not performed and no data available.

The data show that the prepared emulsion formulations (formulation nos. 5, 6 and 7) are physically stable during storage and non-greasy when applied to skin.

### Example 13

The following study was performed to demonstrate the greasy property of the aqueous-based emulsion formulations at Taro Research Institute, New York. Ten human subjects (five females and five males) were randomly selected. The two formulations were each placed in a 2-oz glass jar and labeled as formulation A and formulation B respectively. In each case the human subject was given an ointment comprising urea and salicylic acid (formulation A) and a composition prepared as described in Example 1 (formulation B).

The study was blindfold and human subjects were not allowed to know the content and dosage form of the formulations. The subjects were instructed to apply approximately 250 mg formulation A on one hand and the same amount of formulation B on another hand. The order of which formulations applied on which hand was randomly selected. After the application, the subjects were instructed to rub gently the formulation on the skin for approximately 15-30 seconds. The subjects were then instructed to evaluate if the applied formulation was greasy or non-greasy. Results were recorded in Table 16.

TABLE 16
Physical Characteristics of an Ointment and an Aqueous-Based
Emulsion Containing Urea and Salicylic Acid

| | | | Formulation B |
|---|---|---|---|
| | | Formulation A | (Aqueous-based |
| Human Subject | Gender | (Ointment) | Emulsion) |
| #1 | Male | Greasy | Non-greasy |
| #2 | Male | Greasy | Non-greasy |
| #3 | Male | Greasy | Non-greasy |
| #4 | Male | Greasy | Non-greasy |
| #5 | Male | Greasy | Non-greasy |
| #6 | Female | Greasy | Non-greasy |
| #7 | Female | Greasy | Non-greasy |
| #8 | Female | Greasy | Non-greasy |
| #9 | Female | Greasy | Non-greasy |
| #10 | Female | Greasy | Non-greasy |

It was concluded based on this study that the aqueous-based emulsion formulation was non-greasy as compared to the ointment formulation.

Those skilled in the art will recognize, or be able to ascertain using no more than routine experimentation, many equivalents of the specific embodiments of the invention described herein. The disclosures of the cited publications are incorporated herein in their entireties by reference. It is to be understood, however, that the scope of the present invention is not to be limited to the specific embodiments described above. The invention may be practiced other than as particularly described and still be within the scope of the accompanying claims.

What is claimed is:

1. A stable aqueous-based emulsion formulation, comprising:
(i) from about 8 to about 15 wt % moisturizing agent selected from the group consisting of urea, glycerin, sorbitol and propylene glycol;
(ii) from about 6 to about 15 wt % keratolytic agent selected from the group consisting of salicylic acid and benzoyl peroxide;
(iii) from about 15 to about 50 wt % water;
(iv) from about 2 to about 25 wt % emulsifying agent selected from the group consisting of a mixture of glyceryl stearate and PEG 100 stearate, a mixture of glyceryl stearate and PEG 75 stearate, a mixture of glyceryl stearate and PEG 30 stearate, and polawax; and
(v) from about 0.1 to about 10 wt % stiffening agent selected from the group consisting of cetyl alcohol, stearyl alcohol, cetostearyl alcohol, and cetyl palmitate,
wherein said formulation is non-greasy when applied to skin and exhibits the following characteristics when subject to storage condition of at least about two weeks and at about 35° to about 40° C.:
(1) physical appearance of emulsion does not change;
(2) amount of the moisturizing agent does not change; and
(3) amount of the keratolytic agent does not change.

2. The stable formulation of claim 1, wherein the moisturizing agent is urea.

3. The stable formulation of claim 1, wherein the moisturizing agent is from about 9 to about 12 wt %.

4. The stable formulation of claim 1, wherein the moisturizing agent is about 10 wt %.

5. The stable formulation of claim 1, wherein the keratolytic agent is salicylic acid.

6. The stable formulation of claim 1, wherein the kertaolytic agent is from about 6 to about 9 wt %.

7. The stable formulation of claim 1, wherein the salicylic acid is about 6 wt %.

8. The stable formulation of claim 1, wherein the salicylic acid is micronized salicylic acid having a mean particle diameter of less than 50μ.

9. The stable formulation of claim 1, wherein the salicylic acid is micronized salicylic acid having a mean particle diameter of less than 40μ.

10. The stable formulation of claim 1, wherein the salicylic acid is micronized salicylic acid having a mean particle diameter of less than 25μ.

11. The stable formulation of claim 1, wherein the salicylic acid is micronized salicylic acid having a mean particle diameter of less than 10μ.

12. The stable formulation of claim 1, wherein the salicylic acid is micronized salicylic acid having a mean particle diameter of about 10μ.

13. The stable formulation of claim 1, wherein the water is about 20 to about 45 wt %.

14. The stable formulation of claim 1, wherein the water is about 30 wt %.

15. The stable formulation of claim 1, wherein the mixture of glyceryl stearate and PEG 100 stearate has a wt/wt ratio of about 1:4 to about 4:1.

16. The stable formulation of claim 1, wherein the mixture of glyceryl stearate and PEG 100 stearate has a wt/wt ratio of about 1:1.

17. The stable formulation of claim 1, wherein the mixture of glyceryl stearate and PEG 75 stearate has a wt/wt ratio of about 1:4 to about 4:1.

18. The stable formulation of claim 1, wherein the mixture of glyceryl stearate and PEG 75 stearate has a wt/wt ratio of about 1:1.

19. The stable formulation of claim 1, wherein the mixture of glyceryl stearate and PEG 30 stearate has a wt/wt ratio of about 1:4 to about 4:1.

20. The stable formulation of claim 1, wherein the mixture of glyceryl stearate and PEG 30 stearate has a wt/wt ratio of about 1:1.

21. The stable formulation of claim 1, further comprising a polyoxyethylene 20 sorbitan monoleate.

22. The stable formulation of claim 1, further comprising a sorbitan monostearate.

23. The stable formulation of claim 1, wherein the emulsifying agent is present in the stable formulation of about 5 to about 20 wt %.

24. The stable formulation of claim 1, wherein the emulsifying agent is present in the stable formulation about 15 wt %.

25. The stable formulation of claim 1, wherein the stiffening agent is about 0.5 to about 5 wt %.

26. The stable formulation of claim 1, wherein the stiffening agent is about 2 wt %.

27. The stable formulation of claim 1, further comprising at least one component selected from the group consisting of a stabilizer, emollient, additional emulsifying agent, smoothing agent, humectant, antifoaming agent, oleaginous vehicle, a pH adjuster, and a preservative.

28. The stable formulation of claim 27, wherein the stabilizer is selected from the group consisting of xanthan gum, tragacanth, guar gum, and acacia.

29. The stable formulation of claim 27, wherein the stabilizer is xanthan gum.

30. The stable formulation of claim 27, wherein the emollient is mineral oil light.

31. The stable formulation of claim 27, wherein the additional emulsifying agent is selected from the group consisting of stearic acid, polysorbate 80, polysorbate 20, polysorbate 40, and polysorbate 60.

32. The stable formulation of claim 27, wherein the smoothing agent is cyclomethicone.

33. The stable formulation of claim 27, wherein the humectant is selected from the group consisting of glycerin and propylene glycol.

34. The stable formulation of claim 27, wherein the antifoaming agent is simethicone.

35. The stable formulation of claim 27, wherein the oleaginous vehicle is soybean oil.

36. The stable formulation of claim 27, wherein the pH adjuster is sodium hydroxide.

37. The stable formulation of claim 27, wherein the preservative is selected from the group consisting of benzyl alcohol, methylparaben, propylparaben, ethylparaben sorbic acid, benzoic acid, sodium benzoate, dichlorobenzyl alcohol, and formaldehyde.

38. The stable formulation of claim 27, wherein the preservative is benzyl alcohol.

39. The stable formulation of claim 1, further comprising an anti-fungal agent.

40. The stable formulation of claim 39, wherein the anti-fungal agent is at least one compound selected from the group consisting of amphoteracin B, clotrimazole, econazole, micronazole, terconazole, butoconazole, ticonazole, oxiconazole, sulconazole, ciclopirox olamine, haloprogin, tolnaftate, naftifine, terbinafine, nystatin, and amophotericin B.

41. The stable formulation of claim 39, wherein the anti-fungal agent is terbinafine.

42. The stable formulation of claim 39, wherein the anti-fungal agent is terbinafine hydrochloride.

43. The stable formulation of claim 39, wherein the anti-fungal agent is present in the amount of about 0.1% to about 10%.

44. The stable formulation of claim 39, wherein the anti-fungal agent is present in the amount of about 1% to about 5%.

45. The stable formulation of claim 39, wherein the anti-fungal agent is present in the amount of about 1%.

46. The stable formulation of claim 39, wherein the anti-fungal agent is present in the amount of about 5%.

47. The stable formulation of claim 1, further comprising a corticosteroid.

48. The stable formulation of claim 47, wherein the corticosteroid is at least one compound selected from the group consisting of alclometasone, dipropionate, amcinonide, beclomethasone dipropionate, betamethasone, betamethasone benzolate, betamethasone dipropionate, desonide, desoximetasone, betamethasone valerate, clobetasol propionate, clocortolone pivalate, cortisol, cortisone, fludrocortisone, flunisolide, fluocinonide, prednisone, prednisolone, 6α-methylprednisolone, triamcinolone, dexamethasone, and mometasone furoate.

49. The stable formulation of claim 47, wherein the corticosteroid is desoximetasone.

50. The stable formulation of claim 47, wherein the corticosteroid is present in the amount of about 0.01% to about 5%.

51. The stable formulation of claim 47, wherein the corticosteroid is present in the amount of about 0.1% to about 2%.

52. The stable formulation of claim 47, wherein the corticosteroid is present at about 1%.

53. The stable formulation of claim 1, further comprising an antioxidant.

54. The stable formulation of claim 53, wherein the antioxidant is at least one compound selected from the group consisting of genestein, vitamin C, vitamin E, avobenzone, and octinoxate.

55. The stable formulation of claim 1, further comprising an anesthetic agent.

56. The stable formulation of claim 55, wherein the anesthetic agent is at least one compound selected from the group consisting of lidocaine, and pramoxine.

57. The stable formulation of claim 1, further comprising a vitamin D₃ derivative.

58. The stable formulation of claim 57, wherein the vitamin D₃ derivative is calcipotriene.

59. The stable formulation of claim 1, further comprising an anti-viral agent.

60. The stable formulation of claim 59, wherein the anti-viral agent is at least one compound selected from the group consisting of 5-fluorouracil, imiquimoid, and cidofovir.

61. The stable formulation of claim 1, wherein the formulation is stable for at least about 4 weeks.

62. The stable formulation of claim 1, wherein the formulation is stable for at least about 6 weeks.

63. A stable aqueous-based emulsion formulation, comprising:
(i) from about 8 to about 15 wt % moisturizing agent selected from the group consisting of urea, glycerin, sorbitol and propylene glycol;
(ii) from about 6 to about 15 wt % keratolytic agent selected from the group consisting of salicylic acid and benzoyl peroxide;
(iii) from about 15 to about 50 wt % water;
(iv) from about 2 to about 25 wt % emulsifying agent comprising a mixture of glyceryl stearate, PEG 100 stearate, polawax, tribehenin, cetearyl alcohol, PEG-20 cetyl phosphate, diacetyl phosphate and cetyl palmitate; and
(v) from about 10 to about 40 wt % solubilizer selected from the group consisting of hexylene glycol, Di-PPG2-myreth-10-adipate, and polysorbate 80, wherein said formulation is non-greasy when applied to skin and exhibits the following characteristics when subject to storage condition of at least about two weeks and at about 35° to about 40° C.:
(1) physical appearance of emulsion does not change;
(2) amount of the moisturizing agent does not change; and
(3) amount of the keratolytic agent does not change.

64. The stable formulation of claim 63, wherein the solubilizer is about 30 wt %.

65. The stable formulation of claim 63, wherein the solubilizer is hexylene glycol.

66. The stable formulation of claim 65, wherein the hexylene glycol is about 30 wt %.

67. The stable formulation of claim 63, wherein the solubilizer is a mixture of hexylene glycol and Di-PPG2-myreth-10-adipate.

68. The stable formulation of claim 67, wherein the hexylene glycol is about 15 wt % and Di-PPG2-myreth-10-adipate is about 15 wt %.

69. The stable formulation of claim 63, wherein the solubilizer is a mixture of hexylene glycol and polysorbate 80.

70. The stable formulation of claim 69, wherein the hexylene glycol is about 15 wt % and polysorbate 80 is about 15 wt %.

71. A method for treating a skin condition in a mammal, comprising the step of topical administering a stable aqueous-based emulsion formulation of claim 1.

72. The method of claims 71, wherein the skin condition is ichthyosis.

73. The method of claim 71, wherein the skin condition is xeroderma.

74. The method of claim 71, wherein the mammal is a human.

75. A method for treating a skin condition in a mammal, comprising the step of topical administering a stable aqueous-based emulsion formulation of claim 63.

76. A method for preparing a stable aqueous-based formulation of claim 1, comprising the steps of:
(a) combining an emulsifying agent with a stiffening agent to form a first solution;
(b) dissolving a moisturizing agent in water to form a second solution;
(c) adding the second solution to the first solution to form an emulsion; and
(d) adding a keratolytic agent to the emulsion to form a stable aqueous-based formulation.

77. The method of claim 76, wherein the step a) is performed by heating at a temperature of about 50 to about 90° C.

78. The method of claim 76, wherein the step a) is performed by heating at a temperature of about 60 to about 80° C.

79. The method of claim 76, wherein the step a) is performed by heating at a temperature of about 70° C.

80. The method of claim 76, wherein the step b) is performed by heating at a temperature of about 50 to about 90° C.

81. The method of claim 76, wherein the step b) is performed by heating at a temperature of about 60 to about 80° C.

82. The method of claim 76, wherein the step b) is performed by heating at a temperature of about 70° C.

83. A method for preparing a stable aqueous-based formulation of claim 1, comprising the steps of:
(a) preparing a first solution of emulsifying agent;
(b) dissolving a moisturizing agent in water to form a second solution;
(c) adding the second solution to the first solution to form an emulsion;
(d) adding a keratolytic agent to a solubilizer to form a third solution; and
(e) adding the third solution to the emulsion to form a stable aqueous-based formulation.
